(19) United States
(12) Patent Application Publication
(10) Pub. No.: US 2026/0174599 A1
(43) Pub. Date: Jun. 25, 2026

(54) PUNCH FOR PARTITIONING A CORNEAL GRAFT AND CORRESPONDING BLADE HOLDER

(71) Applicant: MORIA, BOURBON-L'ARCHAMBAULT (FR)

(72) Inventors: Damien GATINEL, Bourbon-l'Archambault (FR); Alain SAAD, BOURBON-L'ARCHAMBAULT (FR)

(21) Appl. No.: 19/128,701

(22) PCT Filed: Nov. 8, 2023
(86) PCT No.: PCT/EP2023/081104

§ 371 Date: May 9, 2025

(30) Foreign Application Priority Data
Nov. 10, 2022 (FR) 2211771

(51) Int. Cl. A61F 9/013

(52) U.S. Cl. CPC A61F 9/013

(57) ABSTRACT

A punch for cutting a corneal graft includes a base having a support surface for corneal tissue and a blade holder provided with at least one blade. The base includes a guide for guiding the blade holder in translation along a central axis normal to the support surface between a first position in which the blade is spaced apart from the support sur-face and a second position in which the blade is adjacent to the support surface. The blade is a blade for partitioning corneal tissue extending substantially in a radial direction with respect to the central axis.

The present invention relates to the field of corneal transplantation, in particular to the field of graft preparation in the context of corneal surgery (or keratoplasty), in particular the preparation of the graft for carrying out the endothelial corneal transplantation technique with a Descemet membrane graft, otherwise known as the DMEK technique (Descemet Membrane Endothelial Keratoplasty).

### BACKGROUND OF THE INVENTION

The cornea corresponds to the transparent anterior portion of the eye; its main function is to protect the interior of the eye from external attacks and shocks and it constitutes the first refractive element of the eye. The cornea is constituted by several transparent layers of tissue which therefore enable light to be transmitted to the lens and the retina.

As a result of external attacks (shock, burns, etc.) or pathological or age-related degeneration, the cornea may thicken and become opaque, to the point of causing blindness. To remedy this, it is possible to carry out a transplant of all or a portion of the cornea.

Several transplantation techniques exist. They are distinguished by the nature and tissue composition of the graft which is removed. Thus, the PK technique (Penetrating Keratoplasty) uses a graft taken from the entire thickness of the donor's cornea (epithelium; Bowman's membrane; stroma; Dua layer; Descemet's membrane; endothelium). The DALK technique (Deep Anterior Lamellar Keratoplasty) uses a graft constituted by the anterior and median layers (epithelium; Bowman's membrane; stroma; Dua layer). More recent techniques, DSAEK and ¹ Translation of the title as established ex officio. DMEK, use grafts respectively constituted by the Descemet membrane and the endothelium, or the Descemet membrane alone.

These last two techniques (DSAEK and DMEK) are currently preferred, because they do not require trephination of the recipient eye, thus enabling the patient to recover more rapidly.

Irrespective of the techniques used, the grafts are removed from corneas of deceased subjects, these corneas being stored in graft banks. Even though there is no incompatibility between the donor and the recipient, the number of available grafts is still insufficient.

In fact, not only is the number of donors not large enough, but also, the techniques for tissue removal then preparation of the graft intended for the recipient are carried out manually and are very delicate, even for experienced people. The removal of the donor tissue comprising the cornea, the preparation thereof for grafting as well as the graft per se, are all steps which are likely to deteriorate the graft, making it unusable, which also contributes to the shortage of grafts.

Thus, safeguarding and assisting in carrying out these steps is the subject of a great deal of research and the development of tools and devices for removing and preparing the grafts.

Among these, mention may be made of tools for assisting in cutting and preparing the graft.

In particular, cutting devices popularly known as “punches” are available on the market.

A punch comprises, on the one hand, a circular base at the centre of which the donor's corneal tissue is held by an automatic or manual suction system (a syringe, for example) connected to the base, and, on the other hand, a blade support which will be fitted around the base, thus enabling the operator (surgeon or graft bank technician) to cut the piece of cornea cleanly in a circular manner to the desired diameter in accordance with the morphology and the indications of the patient to BE transplanted.

Reference may be made to the following documents by way of example: US-A-2021121328, which describes a circular blade having a U-shaped extension; FR-A-2992849, which describes a circular arc-shaped blade; and US2020/0121505, which describes a system for guiding the blade support with respect to the base in order to optimize cutting.

However, those devices have several disadvantages. Thus, even though those devices make it possible to safeguard the preparation of the graft to a certain extent, a single sample of removed donor tissue can only result in a single potential graft. In other words, one cornea removed from one donor can only produce one graft.

In addition, even if the cutting of the graft is guided by these devices, they do not facilitate handling after cutting. In particular, when Descemet membrane grafts are to be made for DSAEK or DMEK surgery, the Descemet membrane, which is both very thin and very elastic, will tend to roll up on itself, making it difficult to put the graft into place in the patient to be transplanted. It is then necessary to unroll the graft before transplantation, without tearing it.

### OBJECTIVE OF THE INVENTION

A particular objective of the invention is to overcome the aforementioned disadvantages, at least in part, by proposing a punch which facilitates the production of corneal grafts.

### SUMMARY OF THE INVENTION

To this end, a punch for cutting corneal graft is provided, having a corneal tissue support surface and a blade holder provided with at least one blade. The base comprises a guide for guiding the blade holder in translation along a central axis normal to the support surface between a first position in which the blade is spaced apart from the support surface and a second position in which the blade is adjacent to the support surface. In accordance with the invention, the blade is a corneal tissue partitioning blade extending substantially along a direction which is radial to the central axis.

It is therefore possible to obtain at least two grafts from the same donor corneal tissue, thereby increasing the number of transplants which can be carried out from the same corneal tissue.

Preferably, the partitioning blade has an S-shaped section along a plane perpendicular to the central axis.

In accordance with a preferred embodiment of the invention, the partitioning blade extends on either side of the central axis in order to divide the corneal tissue into two portions.

Preferably, the blade is disposed in a manner such that the two pieces of corneal tissue obtained from the donor corneal tissue do not mirror each other.

Preferably, the blade is disposed in a manner such that the two pieces of corneal tissue obtained have a portion with an acute angle, which facilitates gripping thereof.

In an alternative embodiment of the invention, the blade holder is provided with at least three partitioning blades, thereby defining an angular sector in pairs.

Thus, the punch in accordance with the invention makes it possible to obtain at least two pieces of corneal tissue comprising at least one end forming an angular sector (in the form of a point and all the better with an acute angle), starting from just one piece of corneal tissue taken from a donor and placed on the support surface of the base of the punch, by fitting into the blade holder. Not only does the punch in accordance with the invention make it possible to obtain several pieces of corneal tissue from a single sample of donor tissue, but it also makes it easier to manipulate. In fact, when the piece of corneal tissue cut from the Descemet membrane rolls up, the presence of an end with an angular sector (in the form of a point and all the better with an acute angle) makes it easier for the operator (surgeon or preparer) to unroll it.

In addition, the blade holder may be provided with a circular peripheral blade centred on the central axis. Preferably, the peripheral blade and the partitioning blade have heights such that the peripheral blade and the partitioning blade penetrate into the corneal tissue to a depth at least equal to a thickness of the Descemet membrane.

Optionally, at least one of the blades of the blade holder is removable.

The guide preferably comprises two complementary guide surfaces, parallel to the central axis, cooperating with one another and with one belonging to the base and the other belonging to the blade holder. The blade holder and the base are advantageously disposed so as to fit into each other.

In addition, the punch in accordance with the invention may comprise indexing means comprising a positioning index secured to one of the base and the blade holder for positioning in a notch provided in the other of the base and the blade holder when fitting together.

The present invention also concerns the blade holder provided with at least one punch partitioning blade as described above. Other features and advantages of the invention will become apparent from reading the following description of particular and non-limiting embodiments of the invention.

### BRIEF DESCRIPTION OF THE DRAWINGS

Reference will be made to the accompanying drawings, in which:

FIG. 1: is a diagrammatic view in axial section of a punch in accordance with a first embodiment of the invention;

FIG. 2: is an enlarged diagrammatic view of box II of FIG. 1;

FIG. 3: is a diagrammatic view in axial section of a blade holder in accordance with a first embodiment of the invention;

FIG. 4: is a diagrammatic bottom view, in slight perspective, of a blade holder in accordance with a first embodiment of the invention, showing the interior of said blade holder;

FIG. 5: is a diagrammatic top view, in slight perspective, of a punch base in accordance with a first embodiment of the invention;

FIG. 6: is a diagrammatic bottom view, in slight perspective, of a blade holder in accordance with a variation of the invention, showing the interior of said blade holder;

FIG. 7: is a diagrammatic axial sectional view of a punch in accordance with a second embodiment of the invention;

FIG. 8: is an enlarged diagrammatic view of box VIII of FIG. 7;

FIG. 9: is a diagrammatic axial sectional view of a blade holder in accordance with a second embodiment of the invention;

FIG. 10: is a diagrammatic bottom view, in slight perspective, of a blade holder in accordance with a second embodiment of the invention, showing the interior of said blade holder;

FIG. 11: is a diagrammatic top view, in slight perspective, of a punch base in accordance with a second embodiment of the invention;

FIG. 12: is a diagrammatic axial sectional view of a punch in accordance with a first variation of a second embodiment of the invention;

FIG. 13: is a diagrammatic view, in slight perspective, of a blade holder in accordance with a second variation of a second embodiment of the invention;

FIG. 14: is a diagrammatic axial sectional view of a punch in accordance with a third embodiment of the invention;

FIG. 15: is an enlarged diagrammatic view of box XV of FIG. 14;

FIG. 16: is a diagrammatic axial sectional view of a punch in accordance with a fourth embodiment of the invention;

FIG. 17: is an enlarged diagrammatic view of box XVII of FIG. 16;

FIG. 18: is a diagrammatic axial section view of a punch in accordance with a variation of a fourth embodiment of the invention;

FIG. 19: is an enlarged diagrammatic view of box XVII of FIG. 18;

FIG. 20: is a diagrammatic view, in slight perspective, of corneal tissue after cutting with a punch in accordance with a first or third embodiment of the invention, in which the piece of cornea C2 has already been removed and the cut piece of cornea C1 is still to be removed from the corneal tissue C;

FIG. 21: is a diagrammatic view, in slight perspective, of corneal tissue after cutting with a punch in accordance with a second or fourth embodiment of the invention, in which the piece of cornea C2 has already been removed and the cut piece of cornea C1 is still to be removed from the corneal tissue C.

### DETAILED DESCRIPTION OF THE INVENTION

The present invention concerns a punch P for cutting grafts from corneal tissue C, comprising a base 1 and a blade holder 2.

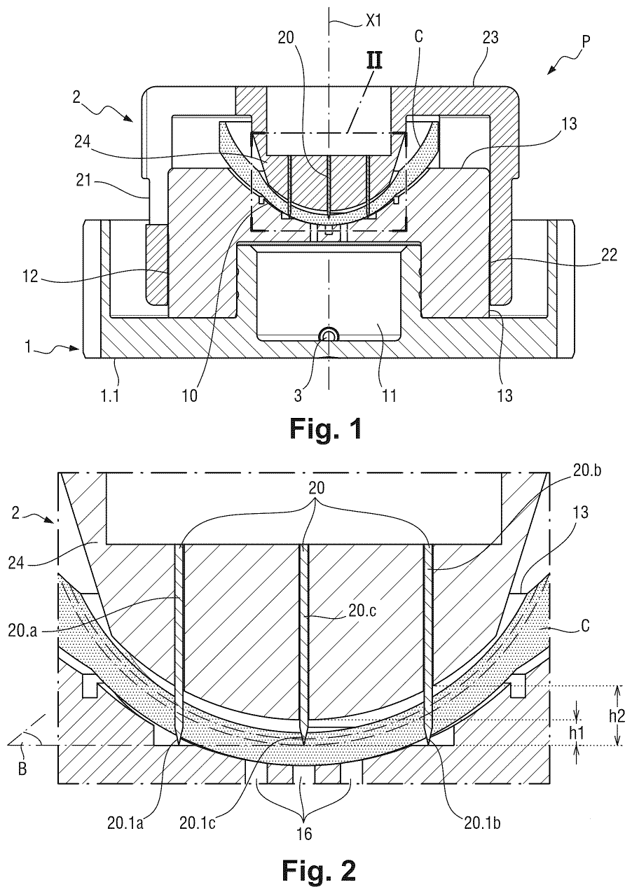

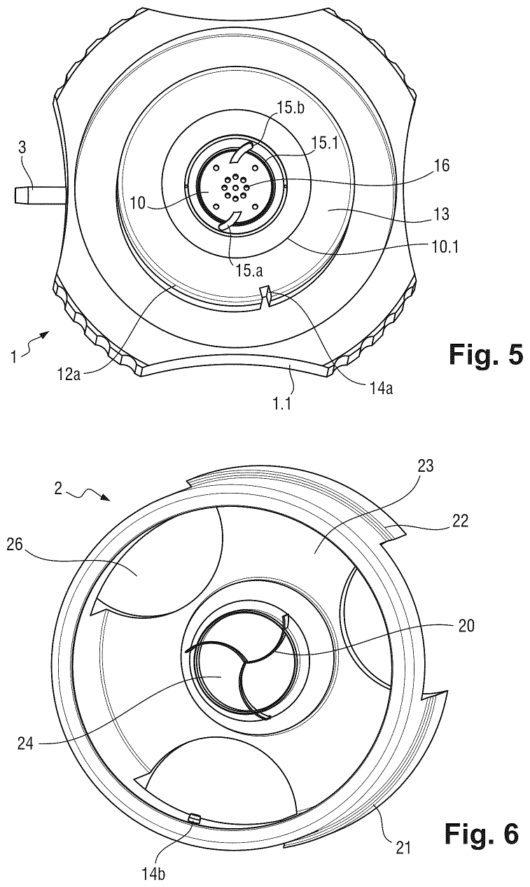

In accordance with a first embodiment of the invention, illustrated in FIGS. 1 to 5, the base 1 comprises a body 1.1 intended to rest on a worktop, on which a receptacle 13 delimiting a closed enclosure 11 with the body 1.1 is fitted. Conventionally, the chamber 11 is connected to a manual or automatic suction device 3. The receptacle 13 of the base 1 further comprises an outer surface forming a cup-shaped support surface 10 which is intended to receive corneal tissue C to be cut and which is in the shape of a body of revolution about a central axis X1. Corneal tissue C is removed from the eye of the donor in accordance with techniques which are known per se. The corneal tissue C has a domed shape with a substantially circular section and comprises all of the constituent cellular layers of the cornea, including the Descemet membrane in the context of DMEK surgery (but only a portion of said constituent cellular layers of the cornea in the case of DSAEK surgery). The support surface 10 therefore has a slightly concave shape to accommodate the donor's convex-shaped corneal tissue C, said support surface being delimited by a circular edge 10.1. Preferably, the support surface 10 is pierced by at least one orifice 16 in communication with the closed chamber 11 so that, when the suction device 3 is actuated, a vacuum is generated at the support surface 10 in order to press and hold the corneal tissue C in place against the support surface 10. The support surface 10 generally has a diameter which is greater than the usual diameter of the corneal tissue C, i.e. generally greater than 10 mm.

The blade holder 2, illustrated more particularly in FIGS. 1, 2, 3 and 4, comprises a cap 21 which is disposed to cover the receptacle 13 of the base 1 and which comprises a sleeve 22 having a central axis X2 and an end partially closed by a bottom 23, said bottom 23 defining the plane A, substantially perpendicular to the axis X2. The bottom 23 is perforated with openings 26 to enable the practitioner to see inside the cap 21 when cutting the corneal tissue C. The cap further comprises a central stud 24 which protrudes out from the bottom 23 in the interior of the sleeve 22. The central stud 24 here has a surface with a slightly convex shape defined so as to be inserted into the slightly concave shape of the surface of the support 10 of the base 1. The central stud 24 is provided with at least one blade 20 which extends substantially in a radial direction with respect to the central axis X2 in order to have a partitioning function. More precisely, the blade 20 has an average plane extending parallel to the central axis X2 and radially with respect thereto. Thus, the blade 20 is intended to divide the corneal tissue C, which rests on the support surface 10, into at least two pieces of cornea C1, C2, in a substantially radial direction with respect to the central axis X2. Each of the two pieces of cornea C1, C2 respectively forms a graft. The blade 20 is fixed in the central stud 24 of the blade holder 2 in a manner such that the cutting edge 20. 1 of the blade protrudes axially from the central stud 24 and in a radial direction with respect to the central axis X2 in order to define a plane B which is substantially parallel to the plane A and therefore perpendicular to the axis X2. The blade 20 may be fixed to the central stud 24 by any means known per se to the person skilled in the art; by way of non-limiting example, this may be fixing by: clamping or squeezing on either side of the blade, by screwing, by overmoulding, by bonding, etc. The blade may be permanently fixed, or the blade may be removable in order to facilitate changing and/or cleaning thereof.

Here, the blade 20 has an S-shaped section in a plane perpendicular to the central axis X2 and preferably extends either side of the central axis X2 in order to divide the corneal tissue C into two portions so as to form two pieces of cornea C1, C2 comprising at least one end forming an angular sector (in the form of a point and all the better with an acute angle). The person skilled in the art will know how to define the length of the blade 20 as a function of the diameter of the corneal tissue to be cut and the radii of curvature Of the S of the blade in a manner such that the blade 20 divides the corneal tissue C in two.

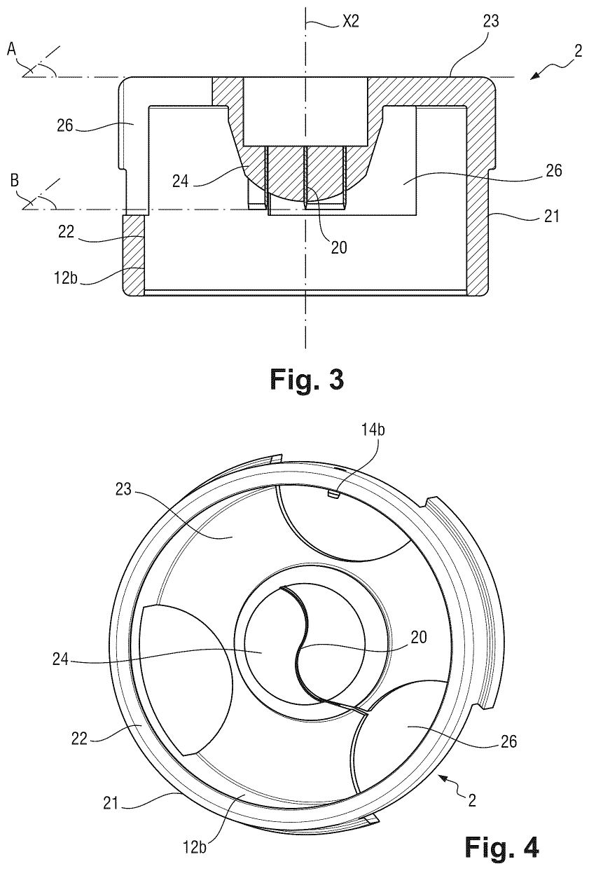

In accordance with this first embodiment of the invention, the cutting edge 20.1 of the blade 20 extends in a plane B substantially parallel to the plane A, defined by the bottom 23, as is particularly illustrated in FIG. 3. The height of the blade 20 protruding from the stud 24 is defined in a manner such that when the blade holder 2 and the base 1 are assembled, the cutting edge 20.1 of the blade 20 penetrates into the corneal tissue C to a depth at least equal to a thickness of the Descemet membrane, i.e. to a depth at least equal to 0.35 mm. The person skilled in the art will know how to determine the height of the blade 20 protruding from the stud 24, as a function of the shape of the stud 24, in particular its convexity, in a manner such that, on the one hand, the cutting edge 20.1 of the blade 20 lies in a plane B parallel to the plane A and that the cutting edge 20.1 penetrates into the corneal tissue C to a depth at least equal to the Descemet membrane. By way of illustration, here, the central portion 20c of the blade protrudes from the convex surface of the stud 24 by a height h1 of 0.35 mm, while the ends 20a and 20b of the blade protrude from the stud 24 by a height h2 of at least 1.1 mm; in this case, therefore, h1 is different from h2.

In accordance with this first embodiment, clearances 15a; 15b may be provided in the receptacle 13 and/or the support surface 10 of the base 1 facing the cutting edge 20.1 of the blade 20 in order to accommodate a portion of each of the ends 20.1a; 20.1b of the partitioning blade 20 in a manner such that the central portion of the blade 20.1c comes into contact with corneal tissue C placed on the concave support 10 of the base 1 in order to cut it into two pieces C1 and C2 over a thickness at least equal to the Descemet membrane.

In order to cut the corneal tissue C into two pieces C1 and C2, the blade holder 2 is assembled on the base 1 by using a guide 12. The guide 12 is arranged so as to guide the blade holder 2 relatively in translation along a central axis X1, X2 normal to the support surface 10 between a first position in which the blade 20 of the blade holder 2 is spaced apart from the support surface 10 and a second position in which the blade 20 is adjacent to the support surface 10. The guide 12 comprises two complementary surfaces parallel to the central axis X1, X2, with one 12a, belonging to the base 1 and the other, 12b, belonging to the blade holder 2. Here, the guide 12 is formed by the outer surface 12a of the receptacle 13 and the inner surface 12b of the sleeve 22 of the blade holder 2. Here, the receptacle 13, which is cylindrical in shape, has a circular section with a slightly smaller diameter than the internal diameter of the sleeve 22 of the blade holder 2.

Here, the punch P further comprises means 14 for angularly indexing the blade holder 2 with respect to the base 1 about the central axis X1, X2 (coinciding when fitted together). The indexing means comprise a positioning index secured to one of the base 1 and the blade holder 2 so as to be placed in a notch provided in the other of the base 1 and the blade holder 2 when fitting together. Here, the positioning index 14b is secured to the blade holder 2. It is a raised element (such as a rib or a lug) protruding from the inner surface of the sleeve 22 of the blade holder 2. This raised element comes into engagement with a notch 14a of the receptacle 13 of the base 1. The person skilled in the art will know how to position the index 14b and the notch 14a in a manner such that the ends 20a, 20b of the partitioning blade 20 come to face the clearances 15a, 15b of the base 1 exactly during assembly of the blade holder 2 with the base 1, therefore enabling a clean cut of the two pieces C1, C2 to be made from the corneal tissue C while preventing a relative rotational movement of the base 1 and the blade holder 2 during assembly. Thus, when the blade holder 2 is engaged on the receptacle 13, the cutting edge 20.1a and 20.1b of the ends 20.a, 20.b of the partitioning blade come to bear on the bottom of the clearances 15.a and 15.b and the cutting edge 20.1c of the central portion 20c of the partitioning blade 20 cuts the corneal tissue C into two pieces C1 and C2 over a height at least equal to the thickness of the Descemet membrane.

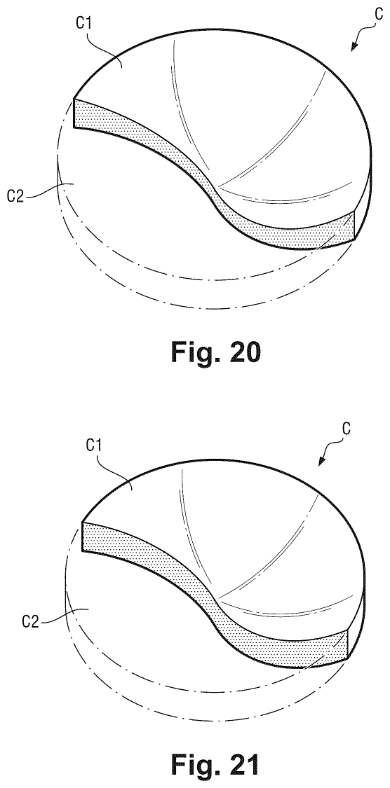

The two pieces of corneal tissue C1, C2 thus obtained are inscribed in the circle formed by the initially removed corneal tissue C; to put it pictorially, the two pieces of corneal tissue C1 and C2 substantially reproduce the two complementary shapes of the known “yin” and “yang” symbol. It should be noted that the S-shaped blade 20 cuts the corneal tissue in a manner such that each piece C1, C2 obtained after partitioning has a point defining an acute angle and, at the opposite end, a wider zone with a more or less semicircular shape. It should also be noted that in accordance with this first embodiment in which the cutting edge 20.1 of the blade 20 defines a plane B, the piece of cornea C1, C2 obtained has a point and an opposite end (of more or less circular shape) which are thicker than the centre of said piece of cornea because the cut is deeper at the periphery than at the centre, as illustrated in FIG. 20.

In accordance with a variation of the first embodiment of the invention, as illustrated in FIG. 6, the blade holder 2 comprises at least three partitioning blades defining an angular sector in pairs. In a manner similar to that of the first embodiment, the person skilled in the art will be able to provide clearances in the base 1 facing the ends of each of the blades in a manner such that, when the blade holder 2 is assembled on the base 1, the ends of the blades come to bear against the clearances and the central portion of the blades cuts out the corneal tissue placed on the support over a thickness at least equal to the Descemet membrane. In the same manner, the person skilled in the art will position the angular indexing means in a manner such that the ends of each of the blades face the clearances provided in the base 1 exactly.

In the following description of other embodiments of the invention, it should be pointed out that elements which are identical or similar to those described above are given the same reference numeral.

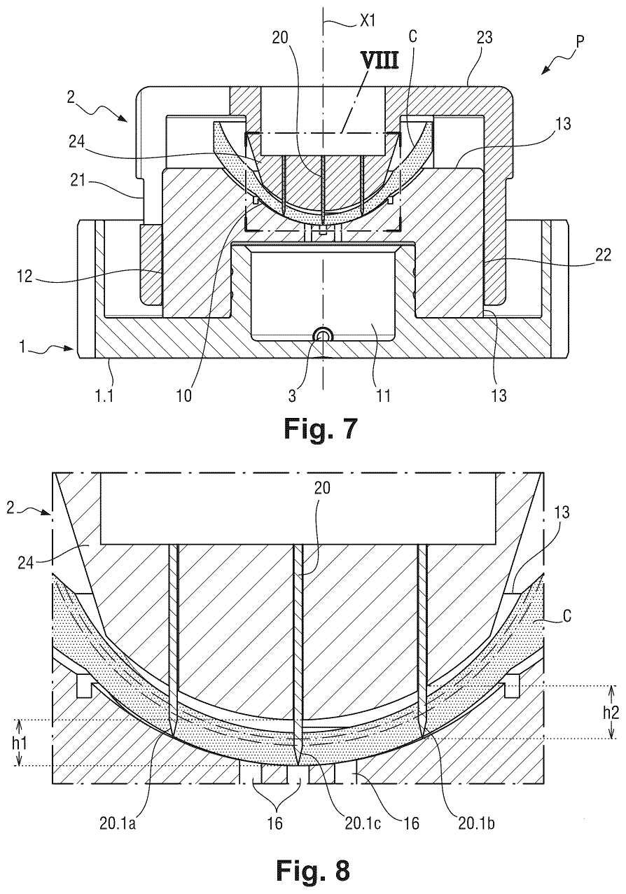

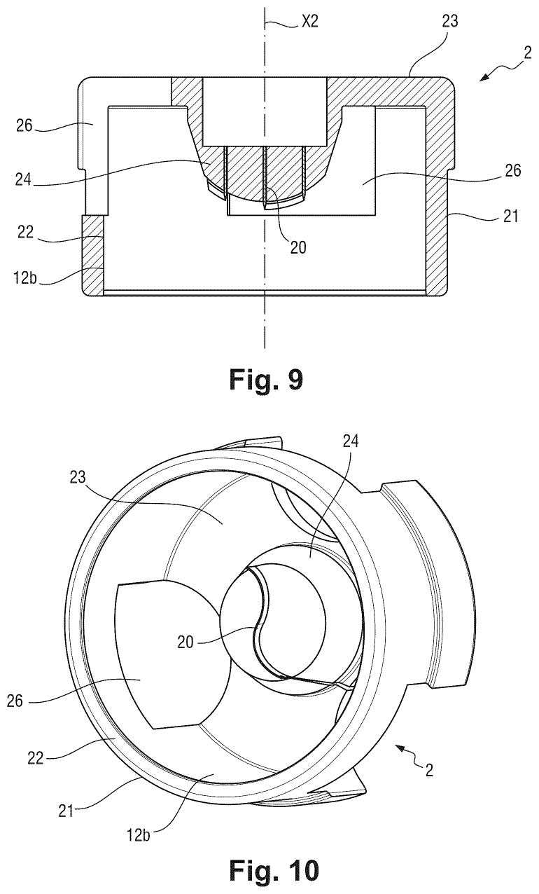

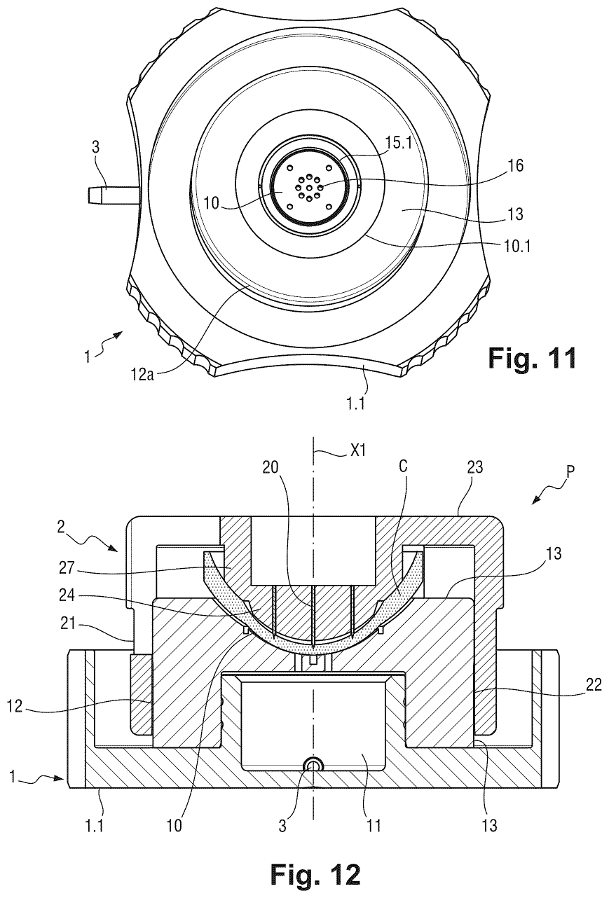

In accordance with a second embodiment of the invention, illustrated in FIGS. 7 to 10, the punch P comprises a base 1 and a blade holder 2. Here, the blade holder 2, particularly illustrated in FIGS. 9 and 10, differs from the blade holder described in the first embodiment of the invention in that the cutting edge 20.1 of the blade 20 protrudes from the surface of the stud 24 over the same height h1, h2 in a manner such that the cutting edge 20.1 of the blade 20 matches the concave shape of the support surface 10. It will be noted that here, in contrast to the first embodiment of the invention, the cutting edge 20.1 of the blade does not define a plane B parallel to the plane A, the height h1 being identical to the height h2. The height h1, h2 is defined in a manner such that when the blade holder 2 is assembled on the base 1, the cutting edge of the blade 20.1 cuts the corneal tissue C into two pieces C1 and C2 over a thickness at least equal to the Descemet membrane, the two pieces C1 and C2 being inscribed in the circle formed by the initially removed corneal tissue C and substantially reproducing the two complementary shapes of the known “yin” and “yang” symbol. Here, the heights h1, h2, are equal. By way of illustration, the blade 20.1 in FIG. 8 is shown as being transfixing (h1, h2 being equal to at least 0.8 mm).

In accordance with this second embodiment, the base 1 differs from the base 1 described in the first embodiment in that it comprises neither clearances nor indexing means. In fact, these latter are useless, since the cutting edge of the blade 20 has a shape which is complementary to the support surface 10. In this second embodiment, the cut of the corneal tissue C is at least equal to the Descemet membrane. As illustrated in FIG. 21, here, the pieces of cornea C1, C2 obtained thus have a point and an opposite end (of more or less circular shape) with the same thickness as their centre, since the cutout is of the same depth.

In accordance with a first variation of the second embodiment of the invention, illustrated in FIG. 12, the blade holder 2 further comprises abutment means 27, also termed a guard. The abutment means 27 are defined in a manner such that when the blade holder 2 is assembled on the base 1, the abutment means come into contact with the periphery of the corneal tissue C placed on the support surface 10 of the base 1, thereby limiting the height of the cut of the corneal tissue C by the cutting edge 20.1 of the partitioning blade 20 to a thickness at least equal to the Descemet membrane. Here, and by way of illustration, the abutment means 27 are disposed around the central stud 24. In this case, the stud 24 has been thickened, but these abutment means could also be obtained by adding a cylindrical wall around the stud 24 which would come into abutment against the surface 10.1 of the receptacle 13. The person skilled in the art will know how to make the abutment means as they see fit.

It should be noted that the base 1 is identical to that described in the second embodiment of the invention and has no clearances. Provision may be made for these abutment means to be adjustable in order to enable the cutting height to be adjusted. By way of example, the abutment means may comprise a nut/locknut assembly screwed onto the blade holder 2 or the base 1.

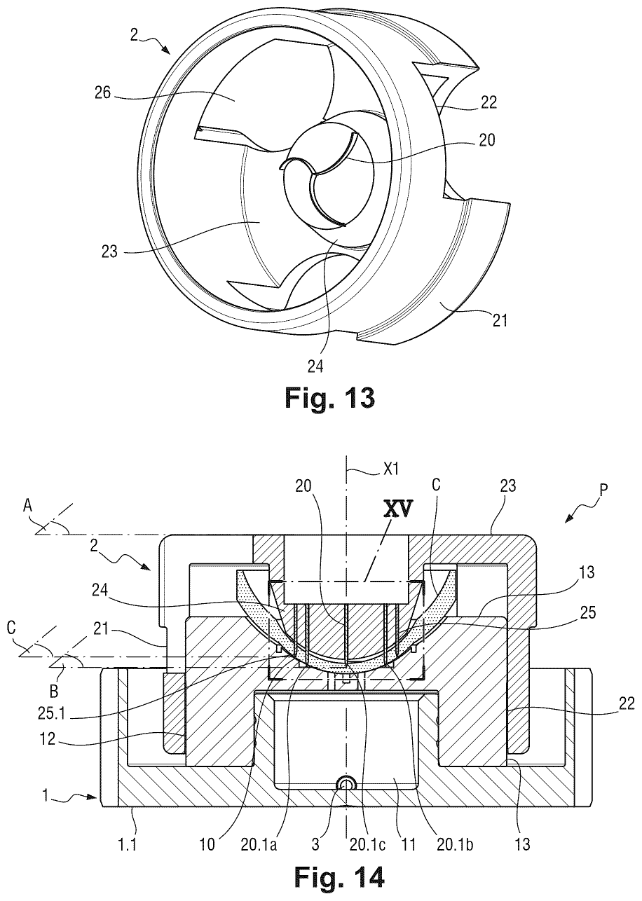

In accordance with a second variation of the second embodiment of the invention, illustrated in FIG. 13, the blade holder 2 comprises at least three blades defining an angular sector in pairs. Here, in analogous manner to the second embodiment of the invention, the cutting edge 20.1 of the blades protrude from the stud 24 over the same height (h1 is equal to h2), the cutting edge of the blades therefore matching the shape of the support surface 10. It should be noted that the blade holder 2 may or may not comprise abutment means 27.

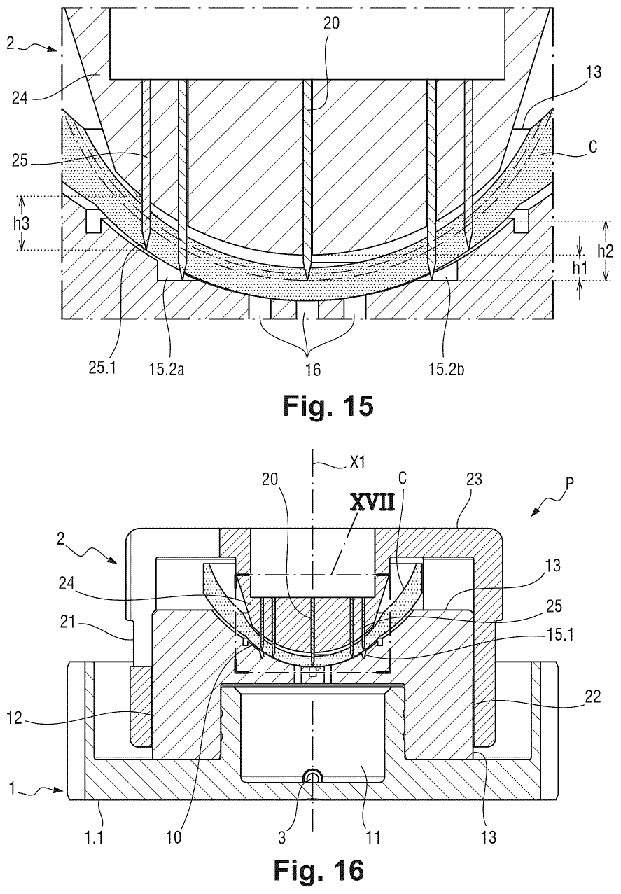

In accordance with a third embodiment of the invention, illustrated in FIGS. 14 and 15, the punch P comprises a base 1 and a blade holder 2.

The blade holder 2 differs from the blade holder 2 described in the first embodiment of the invention in that, in addition to the partitioning blade 20, it comprises a peripheral blade 25 fixed in the central stud 24. The blade holder 2 in accordance with this third embodiment of the invention therefore makes it possible to produce two simultaneous cuts of the corneal tissue C: a circular cut and a cut into two pieces, C1 and C2.

It will be recalled that the cutting edge 20.1 of the partitioning blade 20 defines a plane B parallel to the plane A formed by the bottom 23. The peripheral blade 25, with a circular section, is centred on the central axis X2 and surrounds the partitioning blade 20. The diameter of the peripheral blade 25 is defined as a function of the desired final dimensions of the pieces of corneal tissue C. The person skilled in the art will know how to determine these dimensions as a function of the patient to be grafted. The diameter of the peripheral blade 25 is conventionally between 6 mm and 10 mm. It is possible to provide several blade holders with peripheral blades 25 the diameters of which vary from 10 mm to 6 mm in 0.25 mm increments. The cutting edge 25.1 of the peripheral blade 25 protruding from the surface of the stud 24 defines a plane C parallel to the plane A. The h3 designates the height between the surface of the stud 24 and the cutting edge 25.1 of the peripheral blade. In accordance with this third embodiment, the partitioning blade 20 is substantially inscribed in the peripheral blade 25.

In accordance with this third embodiment, the heights of the cutting edge 20.1 of the partitioning blade 20, h1, h2, and the height h3 of the cutting edge 25.1 of the peripheral blade 25 are defined in a manner such that:
the peripheral blade 25 penetrates into the corneal tissue C to a depth at least equal to the thickness of the Descemet membrane. Preferably, the peripheral blade 25 enables a circular transfixing cut of the corneal tissue C to be produced.
the partitioning blade 20 penetrates into the corneal tissue C to a depth at least equal to a thickness of the Descemet membrane. By way of illustration, the partitioning blade 20 penetrates into the corneal tissue C to a depth of at least 0.35 mm.

Here, as in the first embodiment, the cutting edge 20.1 of the partitioning blade 20 defines a plane B parallel to the plane A and the cutting edge 25.1 of the peripheral blade 25 defines a plane C which is also parallel to the plane A (and therefore to the plane B). Planes B and C may or may not be coincident.

As specified in the first embodiment, the partitioning blade 20 and/or peripheral blade 25 may be permanently fixed to the blade holder 2 or be removable in order to facilitate changing them and/or cleaning the punch.

The base 1 in the third embodiment of the invention is identical to that described in the first embodiment of the invention. Thus, the blade holder 2 and the base 1 are assembled by employing a guide 12, the relative positioning of the blade holder 2 and of the base 1 being achieved by the indexing means 14, the ends 20a, 20b of the partitioning blade 20 coming to bear on the clearances 15a, 15b. The corneal tissue C is thus simultaneously cut in a circle by the peripheral blade 25 and divided into two pieces C1, C2 by the partitioning blade 20 over a thickness at least equal to the Descemet membrane. The two pieces of corneal tissue C1, C2 thus obtained are inscribed in the circle cut out by the peripheral blade 25 in the initially removed corneal tissue C. The pieces of corneal tissue C1, C2 substantially reproduce the two complementary shapes of the known “yin” and “yang” symbol. It should be noted that the blade 20 is shaped in a manner such that each piece of corneal tissue C1, C2 thus obtained has a point defining an acute angle and, at the opposite end, a wider zone with a more or less semicircular shape which is thicker than the central portion, since the cut is deeper at the periphery, as shown in FIG. 20. The pieces of corneal tissue C1, C2 obtained by this third embodiment have dimensions which are useful for direct transplantation onto the eye of a patient.

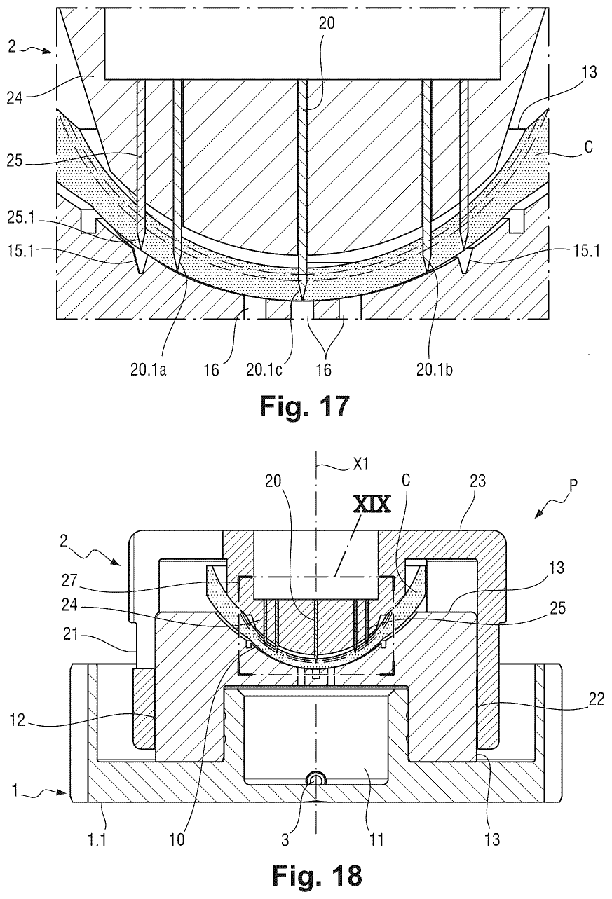

In accordance with a fourth embodiment of the invention, illustrated in FIGS. 16 to 19, the punch P comprises a blade holder 2 which differs from the blade holder described in the second embodiment of the invention in that, in addition to the partitioning blade 20, it comprises a peripheral blade 25 fixed in the central stud 24. It will be recalled that the cutting edge 20.1 of the blade 20 protrudes from the surface of the stud 24 over the same height h1, h2 in a manner such that the cutting edge 20.1 matches the concave shape of the support surface 10, or in other words that the cutting edge 20.1 of the blade follows the convex surface of the stud 24, h1 being equal to h2. As previously described in the third embodiment, the peripheral blade 25, with a circular section, is centred on the central axis X2 and surrounds the partitioning blade 20, its diameter being defined by the person skilled in the art in accordance with the desired final dimensions for the pieces of corneal tissue C1, C2 to be transplanted, conventionally between 6 mm and 10 mm.

The height h3 of the cutting edge 25.1 of the peripheral blade 25 is defined in a manner such that it penetrates into the corneal tissue C to a depth at least equal to a thickness of the Descemet membrane. Preferably, the peripheral blade 25 penetrates into the corneal tissue C over the entire thickness in a manner such that it makes a transfixing cut. The height of the cutting edge 20.1 of the partitioning blade 20 is determined in a manner which is identical to that described in the second embodiment of the invention, in a manner such that the cutting edge 20.1 of the partitioning blade 20 penetrates into the corneal tissue C to a depth at least equal to a thickness of the Descemet membrane. By way of illustration, the partitioning blade 20 penetrates into the corneal tissue C to a depth of at least 0.35 mm. Here, h3 is greater than or equal to h1, h2.

The base 1 in the fourth embodiment of the invention is identical to that described in the second embodiment of the invention and comprises neither clearances nor indexing means. Thus, the blade holder 2 and the base 1 are assembled by using a guide 12, until the peripheral blade 25 and the partitioning blade 20 come to cut the corneal tissue C into two pieces C1, C2 over a depth at least equal to the Descemet membrane. The two pieces C1 and C2 are inscribed in the circle cut out by the peripheral blade 25 in the initially removed corneal tissue C and substantially reproduce the two complementary shapes of the known “yin” and “yang” symbol. It should be noted that the blade 20 is shaped in a manner such that each piece of corneal tissue C1, C2 thus obtained has a point defining an acute angle and, at the opposite end, a wider zone with a more or less semicircular shape. The pieces of corneal tissue C1, C2 obtained by this fourth embodiment have a substantially constant thickness (since the depth of cut is substantially constant), as illustrated in FIG. 21, and dimensions which are useful for direct transplantation onto the eye of a patient.

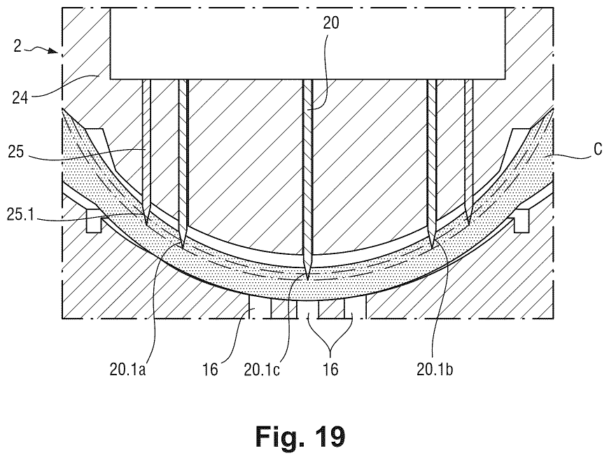

In accordance with a variation of the fourth embodiment of the invention which is analogous to the first variation of the second embodiment, illustrated in FIGS. 18 and 19, the blade holder 2 further comprises abutment means 27, also termed a guard. The abutment means 27 are defined such that when the blade holder 2 is assembled on the base 1, the abutment means come into contact with the periphery of the piece of cornea, thereby limiting the height of the cut of the corneal tissue C by the peripheral blade 25 and by the partitioning blade 20 to a thickness at least equal to the Descemet membrane. The base 1 is identical to that described above. The assembly of the blade holder 2 with the base 1 is also identical, as is the shape of the pieces of corneal tissue C1, C2 thus obtained.

The examples below will provide a better understanding as to how a person skilled in the art will prepare a graft intended for a patient undergoing surgery using the DMEK technique.

### EXAMPLE 1

### Use of a Punch P in Accordance With the First or the Second Embodiment of the Invention for the Preparation of a DMEK Graft

### Step 1: Removal

The corneal tissue is removed from the eye of the donor and placed on the support surface 10 of the base 1.

The suction system 3 is started up in order to “fix” the corneal tissue on the support surface 10.

### Step 2: Scoring

The operator cuts out the corneal tissue C placed on the support surface 10 substantially in a circle with a commercially available blade holder with a peripheral blade, or manually with a hook in order to determine the cleavage plane for the delamination step.

### Step 3: Delamination

Once the cleavage plane has been determined, the operator delaminates the superior tissue layer of the corneal tissue corresponding to the Descemet membrane with the aid of a spatula. This very thin layer (of the order of 12 μm) nevertheless continues to adhere to the other cellular layers of corneal tissue.

### Step 4: Circular Trephination

The operator then cuts out a circle of the delaminated corneal tissue with a blade holder with a peripheral blade of the desired final size, depending on the patient. The cutout here has a diameter of less than 10 mm. In accordance with their usual practice, the operator will either use a blade holder with a guarded blade to make a cut to a height at least equal to the thickness of the Descemet membrane, or will use a blade holder with a peripheral blade, making it possible to make a transfixing cut.

### Step 5: Asymmetric Partition Trephination

The operator then assembles the blade holder 2 of the invention comprising the partitioning blade 20 as described in the first embodiment (cutting edge 20.1 of the blade defining a plane) or as described in the second embodiment (cutting edge 20.1 of the blade substantially matching the concave shape of the support surface) on the base 1 in order to cut the corneal tissue C into two pieces C1, C2.

The operator will select the appropriate base 1 with or without clearances, depending on the selected blade holder.

At the end of this step, the pieces C1, C2 of corneal tissue have the desired dimensions for transplanting. They have the two complementary forms of the known “yin” and “yang” symbol inscribed in the circle cut by the peripheral blade.

### Step 6: Removal of Corneal Tissue Pieces C1, C2

The operator grasps the angular end of the first piece C1 of corneal tissue with the aid of an instrument in order to place it in the preservation liquid or to transplant it directly onto the patient. The same will then be done with the second piece C2 of corneal tissue.

The pieces C1 and C2 thus obtained are perfectly suitable for transplantation in accordance with the DMEK technique, while facilitating manipulation and limiting their number.

### EXAMPLE 2

### Use of a Punch P in Accordance With the Third or Fourth Embodiment of the Invention for the Preparation of a DMEK Graft

Steps 1 to 3 are identical to those described in Example 1.

### Step 4: Simultaneous Circular Trephination and Asymmetric Partitioning

The assembly of the blade holder 2 comprising a peripheral blade 25 and a partitioning blade 20 (cutting edge 20.1 of the blade defining a plane B) as described in the third embodiment or a peripheral blade 25 and a partitioning blade 20 (cutting edge 20.1 of the blade matching the concave shape of the support surface) as described in the fourth embodiment of the invention makes it possible to obtain two pieces of corneal tissue C1, C2 in a single operation and simultaneously, cut by the partitioning blade 20 having the two complementary shapes of the known “yin” and “yang” symbol and inscribed in the circle cut by the peripheral blade 25 to a thickness at least equal to the Descemet membrane.

At the end of this step, the pieces C1, C2 of corneal tissue have the desired dimensions for transplantation.

### Step 5: Removal of Corneal Tissue Pieces C1, C2

This step is identical to step 6 described in the first example. It should be noted that the operator will use the base with or without clearance, depending on the blade holder which is selected.

Thus, the pieces of corneal tissue C1 and C2 have dimensions and a thickness equal to the Descemet membrane which can be used directly for carrying out a transplant in accordance with the DMEK technique.

Thus, the use of a punch comprising a blade holder in accordance with one of the described embodiments of the invention makes it possible to obtain several grafts from a single corneal tissue while limiting the number of manipulations and making them easier. In fact, not only is the corneal tissue C not displaced and is held on the support surface of the base throughout the cutting steps, but in addition, the shape of the pieces of corneal tissue comprises an end forming an acute angle, which makes t possible to orientate the graft, to facilitate unrolling it if necessary, and to improve positioning during the transplantation. In addition, the operator benefits from great versatility in use, in that the grafts can be cut out in accordance with the quality of the corneal tissue, practices and the planned surgery: using one or more blade holders, with a partitioning blade with a cutting edge in the same plane or with a cutting edge substantially matching the shape of the support, or with a peripheral blade and a partitioning blade with a cutting edge in the same plane or with a cutting edge substantially matching the shape of the support, with or without a guard, etc.

Clearly, the invention is not limited to the embodiments described, but encompasses any variation falling within the scope of the invention as defined by the claims.

In particular, the partitioning blade here is pre-formed into the shape of an S, but it may be straight and elastically deformed with a needle after it has been put into place in the central stud 24.

The partitioning blade may also have another shape, and in particular a vertical planar shape.

The blade holder may be provided with several partitioning blades disposed in star shapes, as in FIG. 6, the partitioning blades delimiting angular sectors in pairs.

The person skilled in the art is capable of defining the optimum length of the cutting edge 20.1 of the blade 20 as a function of whether the blade holder 2 comprises one or more partitioning blades 20 and whether or not it comprises a peripheral blade. In fact, if the blade holder comprises a blade 20 extending either side of the central axis X2 in a radial direction, the length of the cutting edge of the blade 20 will be substantially greater than the diameter of the piece of cornea placed on the support surface 10 of the base 1 and centred on the axis X1 (thus, the length of the cutting edge of the blade 20 will vary from substantially 6 mm to a little more than substantially 10 mm). If the blade holder comprises at least three blades 20, the length of each of the blades 20 will be substantially greater than the radius of the piece of cornea placed on the support surface of the base and centred on the central axis X1. Thus, the length of each blade will vary from substantially 3 mm to a little more than substantially 5 mm.

The stud 24 may be shaped in a manner such that it does not to come into contact with the corneal tissue C placed on the support surface 10 when the blade holder 2 is assembled on the base 1 or come into abutment on the outer periphery of the corneal tissue C in order to keep intact the portion of the corneal tissue intended to be cut in order to form the two pieces of cornea C1 and C2.

Here, the stud 24 has a convex surface, but it may also have a planar surface. The person skilled in the art will then know how to adapt the abutment means and the height of the partitioning blade.

In accordance with the first variation of the second or fourth embodiment of the invention, the blade holder comprises abutment means 27 which come to bear on the periphery of the piece of cornea C, but the abutment means 27 may also be defined so as to come to bear on the circular edge 10.1 of the support surface 10 of the base 1.

The abutment means may alternatively be provided on the base.

The base 1 and the blade holder 2 may be produced from one or more easily sterilizable materials such as stainless steel or other stainless steel grades, or from a plastic material having the same characteristics, in particular sterilization capability.

Here, the guide is formed by the surfaces, respectively the outer surface of the receptacle 12a of the receptacle 13 and the inner surface 12b of the sleeve 22 of the blade holder 2, which come to face each other when fitting together, but the guide may also comprise guideposts secured to the base 1 and on which the blade holder 2 slides.

Here, the blade holder 2 will fit on the base 1, but the reverse is also possible.

The punch here has an enclosure which is more or less cylindrical in shape, the blade holder and the receptacle having a cylindrical shape; the body, the blade holder and the receptacle may have any other shape so that the blade holder and the base can be assembled.

The punch in accordance with the invention enables at least two grafts to be obtained from one piece of cornea C intended for keratoplasties of the DSAEK and DMEK type, but it is possible to define the height and shape of the partitioning blade 20 so as to obtain grafts intended for other, DALK or even PK, types of surgery.

1. A punch for cutting a corneal graft, comprising a base having a support surface for corneal tissue and a blade holder provided with at least one blade, the base comprising a guide for guiding the blade holder in translation along a central axis normal to the support surface between a first position in which the blade is spaced apart from the support surface and a second position in which the blade is adjacent to the support surface, wherein the blade is a blade for partitioning corneal tis-sue extending substantially in a radial direction with respect to the central axis and having an S-shaped section in a plane perpendicular to the central axis.

2. The punch as claimed in claim 1, wherein the partitioning blade extends on either side of the central axis in order to divide the corneal tissue into two portions.

3. The punch as claimed in claim 1, in wherein the blade holder is provided with at least three partitioning blades defining an angular sector in pairs.

4. The punch as claimed in claim 1, wherein the blade holder is provided with a peripheral blade centred on the central axis.

5. The punch as claimed in claim 4, wherein the peripheral blade and the partitioning blade have heights such that the peripheral blade penetrates into the corneal tissue to a depth equal to a total thickness of said corneal tissue, the partitioning blade penetrating into the corneal tissue to a depth at least equal to a thickness of the Descemet membrane.

6. The punch as claimed in claim 4, in which wherein at least one of the blades is removable.

7. The punch as claimed in claim 1, wherein the guide comprises two complementary guide surfaces, parallel to the central axis, cooperating with each other and with one belonging to the base and the other belonging to the blade holder.

8. The punch as claimed in claim 7, wherein the blade holder and the base are disposed so as to fit into each other.

9. The punch as claimed in claim 8, comprising means for angularly indexing the blade holder with respect to the base, the indexing means comprising a positioning index integral with one of the base and the blade holder for positioning in a notch provided in the other of the base and the blade holder when fitting together.

10. A blade holder provided with at least one partitioning blade, for the punch as claimed in claim 1.
